(19) United States
(12) Patent Application Publication
(10) Pub. No.: US 2022/0189023 A1
(43) Pub. Date: Jun. 16, 2022

(54) SYSTEMS, METHODS AND COMPUTER PROGRAM PRODUCTS FOR DETECTING INDIVIDUALS WITH FEVER IN VIEW OF ACUTE INFECTION OF THE INDIVIDUAL

(71) Applicant: FEV Group GmbH, Aachen (DE)

(72) Inventors: Sherif Matta, Rochester Hills, MI (US); Qusay Alrousan, Auburn Hills, MI (US)

(21) Appl. No.: 17/117,508

(22) Filed: Dec. 10, 2020

(51) Int. Cl. G06T 7/00; H04N 5/33; H04N 7/18; G08B 21/18; G01J 5/00; A61B 5/01; A61B 5/00

(52) U.S. Cl. CPC G06T 7/0014; H04N 5/33; H04N 7/18; G08B 21/182; G01J 5/0025; A61B 5/015; A61B 2576/00; A61B 5/0077; A61B 5/0035; G06T 2207/10048; G06T 2207/20221; G06T 2207/30196; G06T 2207/30004; A61B 5/0075

(57) ABSTRACT

A method for detecting a plurality of persons with fever including acquiring a first image by a camera, the first image including a first image data of at least a first person, acquiring a second image by a thermal imaging camera, wherein the second image includes a second image data of the first person and a temperature reference apparatus in an infrared spectrum, the second image, at least in sections, has an identical image area as the first image, superimposing the first image and the second image to form a third image, wherein the third image includes a third image data of the first image and the second image, determining a first measurement point within the first image data of the first image, and determining a first temperature for the first measurement point based on the third image and the second image data of the temperature reference apparatus.

### TECHNICAL FIELD

The present disclosure generally relates to detecting a fever in individuals, and more specifically, to systems, methods and computer program products for detecting persons with fever, in view of an acute infection of the persons.

### BACKGROUND

It is generally known to perform a fever triage. Generally, a thermal image-detecting unit is used to detect a testee at a time-point and a position so as to form thermal image data and a visible light image-detecting unit is used to detect the testee at the same time-point and position so as to form visible light image data. The thermal image data is analyzed to determine if at least one fever-temperature block thereof is within a predetermined temperature range.

### SUMMARY

An inventive method for detecting persons with fever, in view of an acute infection of the persons, for access control for secured areas includes the steps of acquiring a first image by means of a camera, said first image includes image data of at least a first person in a visible spectrum, acquiring a second image by means of a thermal imaging camera, wherein the second image includes image data of the first person and a temperature reference apparatus in an infrared spectrum, and wherein the second image at least in sections has an identical image area as the first image, superimposing the first image and the second image to form a third image based on the identical image area, wherein the third image includes image data of the first image and the second image, determining a first measurement point within image data of the first person of the first image and determining a first temperature for the first measurement point based on the third image and the image data of the temperature reference apparatus.

By determining a temperature for the first measurement point based on the first and second image, it is possible to provide a non-contact fever detection of the first person and other persons in the image area of the first and second image.

Especially with regards to acute infections, for example with Covid 19, flu, or any other contagious infection the detection of fever in persons is important and makes it possible to slow down and/or prevent the spread of those infections.

The first image and/or the second image include image data of faces of the persons.

The first person is identified using a database. The database includes identifications of persons and images of those persons.

Identification includes, for example, names and/or addresses of the persons. The fact that names and/or addresses are included in the database makes it possible to trace chains of infection.

The first person is denied access to a secured area if the first temperature is above a threshold temperature. This makes it possible to protect people who are inside the secured area from being infected by the first person.

An inventive apparatus for detecting persons with a fever in view of an acute infection of the persons for access control to secured areas includes a camera for capturing a first image. The first image includes image data of at least a first person in a visible spectrum. The apparatus includes a thermal imaging camera to capture a second image. The second image includes image data of the first person and a temperature reference apparatus in an infrared spectrum. The second image has an identical image area as the first image, at least in sections. The apparatus includes a temperature reference apparatus. The temperature reference apparatus is located within an image area of the second image. The apparatus includes a computing apparatus. The computing apparatus includes a processing device, and a non-transitory, processor-readable storage medium in communication with the processing device, wherein the non-transitory, processor-readable storage medium includes one or more programming instructions.

The one or more programming instructions, when executed cause the processing device to superimpose the first image and the second image to form a third image based on the identical image area. The third image includes image data of the first image and the second image, and/or for determining a first measuring point within the image data of the first person, and/or for determining a first temperature for the first measuring point on the basis of the third image and the image data of the temperature reference apparatus and/or for creating a fourth image, wherein the fourth image includes image data of the first person and a representation of the first temperature.

A computer program product according to the embodiments herein includes a program which, when executed by a computer, causes the computer to perform a method according to the present disclosure for detecting persons with a fever, in view of an acute infection of the person, for access control to secured areas.

The advantages of the method according to the invention arise in the same way for the apparatus according to the invention and the computer program product according to the invention.

Embodiments are explained in more detail based on the following figures.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically illustrates an embodiment of an apparatus and an embodiment of a computer program product according to one or more embodiments shown and described herein;

FIG. 2 schematically illustrates an example of a process according to one or more embodiments shown and described herein; and

FIG. 3 graphically depicts a flowchart of an example method for detecting persons with fever according to one or more embodiments described and illustrated herein.

### DETAILED DESCRIPTION

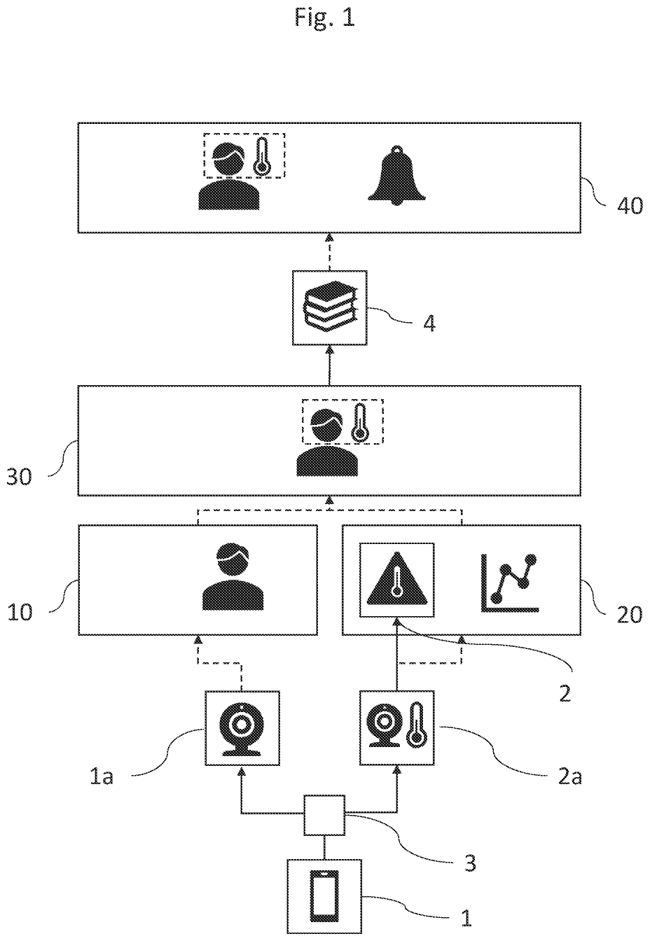

FIG. 1 shows an embodiment of an apparatus according to the invention for the detection of persons with fever in view of an acute infection of those persons for access control to secured areas.

In this example, the apparatus is designed as a smartphone. The smartphone is positioned in an entrance area of a company.

The apparatus includes a camera 1a to acquire a first image 10. The camera 1a is integrated into the smartphone. This enables low production costs for the setup. The fact that smartphones are widely used allows for a widespread use of the apparatus according to the invention.

The camera 1a is positioned in such a way that it can completely capture an entrance door of the entrance area. The entrance door is the only entrance door to the company premises. This makes it possible that every person who enters the company is captured by the camera 1a. Alternatively, several entrance doors are available. Each of these is equipped with an embodiment of an apparatus that complies with the invention.

In a first step, camera 1a acquires a first image 10, which includes image data of at least one first person in a visible spectrum.

In this embodiment, the first image contains 10 image data of a face of the first person. The face of the first person is partially covered with a mouth-nose cover. The first person is not wearing glasses. Accordingly, skin of an area of a forehead of the first person, which is located between two eyes of the first person, is visible to the first camera 1a.

The apparatus includes a thermal imaging camera 2a to capture a second image 20.

The thermal imaging camera 2a is designed separately from the smartphone. The thermal imaging camera 2a is connected to the smartphone by a USB cable and positioned directly adjacent, below the smartphone. The smartphone and the thermal imaging camera 2a are placed on a tripod. This allows a trouble-free acquisition of the first image 10 and the second image 20.

The thermal imaging camera 2a allows the acquisition of 19200 pixels for the second image 20. The temperature range of the thermal imaging camera 2a is between −20° C. and 400° C. Each of the 19200 pixels has a temperature resolution of 0.1° C. In this design example the thermal imaging camera 2a is designed as model FLIR One.

The apparatus includes a temperature reference apparatus 2, which is separate from the smartphone and the thermal imaging camera 2a. The temperature reference apparatus 2 is connected to the thermal imaging camera 2a by a wireless connection. This allows the temperature reference apparatus 2 to be easily positioned so that it can be captured from image areas of the first image 20.

The temperature reference apparatus 2 includes an object with a selectable and stable temperature. The thermal imaging camera 2a uses this temperature to perform a calibration. This calibration enables the measured temperatures to be converted into real temperatures.

Furthermore, it is possible that the temperature reference apparatus 2 transmits the selectable and stable temperature of the object directly to the thermal imaging camera 2a for the purpose of calibration of the thermal imaging camera 2a.

Alternatively, the selectable and stable temperature is entered manually into the thermal imaging camera 2a. This makes it possible to operate without a wireless connection between the thermal imaging camera 2a and the temperature reference apparatus 2.

The thermal imaging camera 2a captures a second image in a subsequent step.

The second image 20 includes image data of the first person and the temperature reference apparatus 2 in an infrared spectrum. The second image 20 has an identical image area as the first image 10, at least in sections.

In this embodiment, the image area of the first image 10 corresponds for more than 90% to the image area of the second image 30. This makes it possible to capture a large number of people with the first image 10 and the second image 20 simultaneously. In this embodiment, the image data of the first image 10 and the second image 20 completely cover the face of the first person.

In a further step, the first image 10 and the second image 20 are superimposed to form a third image 30 by the apparatus. The superimposition is done using the identical image area. The third image 30 includes image data of the first image 10 and the second image 20.

In this embodiment, the third image 30 includes image data of the face of the person in the visible and infrared spectrum.

The superimposition is done by matching pixel groups in the first image 10 and in the second image 20 that refer to matching image areas and/or by using a second-degree function which assigns pixels of the second image 20 to pixels of the first image 10.

A face recognition algorithm is used to determine the face of the first person in the first image 10 and in the second image 20. Based on the matching image areas, the face of the first person is reliably recognized in the first image 10 and in the second image 20.

Using a second-degree polynomial, the pixels of the first image 10 are matched with the pixels of the second image 20. This results in the superimposed third image 30.

In a further step, a first measuring point within the image data of the first person of the first image 10 is determined by the apparatus.

In addition to the face of the first person, the used face recognition algorithm also determines the position of the eyes. A distance between the centers of the two eyes is determined. The first measuring point is located in the middle of the determined distance. This first measuring point is determined based on pixel coordinates. These pixel coordinates are then located in the third image 30.

In a further step, a first temperature for the first measuring point is determined by the apparatus using the third image 30 and the image data of the temperature reference apparatus 2.

Since the temperature reference apparatus 2 is located within the image area of the second image 20, the determined temperature at the first measuring point corresponds to an actual temperature of the face of the first person between the eyes at the time of capturing the second image 20.

In a further step, a fourth image 40 is created by the apparatus. The fourth image 40 includes image data of the first person and a representation of the first temperature.

In this embodiment, the recognized face of the first person in the fourth image 40 is marked with a rectangle. The rectangle protrudes above the recognized face. Within the area that protrudes over the face, the temperature is displayed in Arabic numerals with the corresponding unit.

In a further step, the fourth image 40 is saved by the apparatus if the first temperature is above a threshold temperature.

In this embodiment, the threshold temperature is set manually and is 37° C. The determined temperature of the first measuring point is 37.5° C. Accordingly, the fourth FIG. 40 is stored. This allows to see at a later time which temperature the first measuring point had.

In a further step, the apparatus denies access of the first person to a secured area if the first temperature is above a threshold temperature.

In a further step an alarm is triggered by the apparatus if the first temperature is above threshold temperature.

In this embodiment, the smartphone is illuminated red. This alerts a person working at the reception desk, who then contacts the first person for further instructions for the first person.

Alternatively, all employees of the company authorize access to the company by smart cards. In this case this authorization would be denied.

In a further step, the first person is identified by means of a database 4, which contains identification of persons and pictures of the persons.

The database 4 contains pictures of the faces of all employees, the related supervisors, the workplaces and the private addresses of the employees.

The face recognition algorithm compares the recognized face of the first person with the images from database 4. If a match is found, the first person is identified as the person found.

In a further step, a responsible authority for the first person is determined using database 4.

In this embodiment the superior of the first person is determined as the responsible authority.

Simultaneously with the previous step, the alarm is triggered at the responsible supervisor by the apparatus.

In this embodiment an e-mail is sent to the superior by the institution. This enables the supervisor to initiate further measures.

As an alternative to this example, the apparatus is used for access control of a public building and/or vehicle. Other alternative applications include access control of stadiums, airports, event sites, hotels, hospitals, stores with or without public traffic, offices and/or workplaces, and military and/or civil checkpoints.

In some embodiments, the apparatus includes a computer program product 3. The computer program product 3 includes a program which, when executed by a computer, causes the computer to execute an embodiment of a process according to a method according to the embodiments described herein.

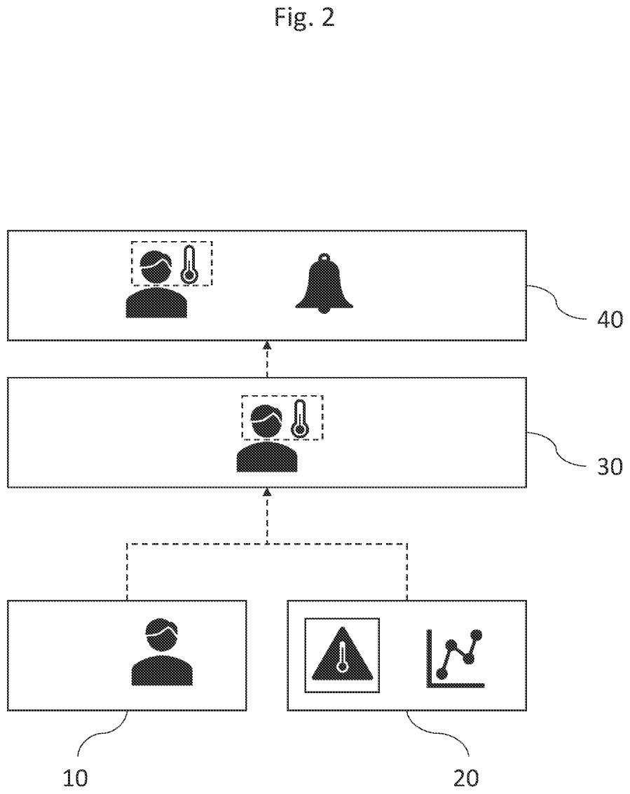

FIG. 2 schematically illustrates an embodiment of a method according to the invention for the detection of persons with fever, in view to an acute infection of the persons, for access control for secured areas.

In this embodiment, the first image 10 includes further persons in addition to the first person.

In this embodiment a total of three persons are included. Therefore, a second and a third person are captured in addition to the first person. The steps described in the first embodiment are performed separately for all three persons in this embodiment.

This enables a large number of persons to be checked simultaneously for fever and/or an elevated temperature when they enter a secured area.

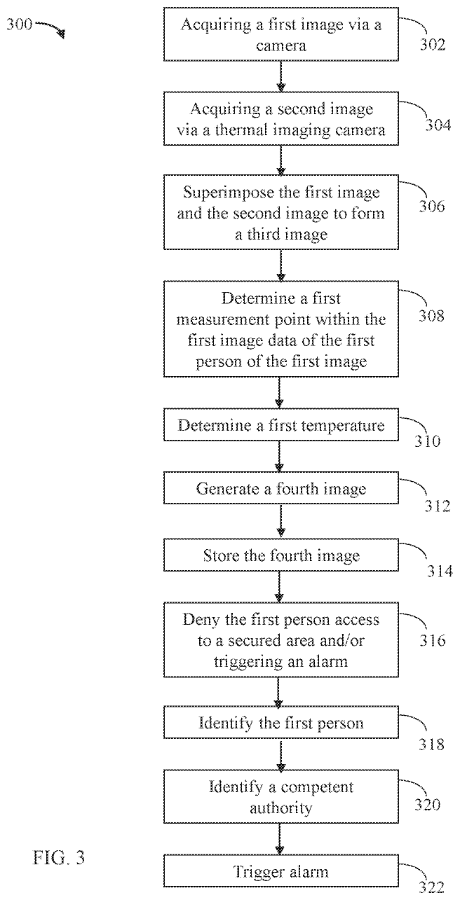

Now referring to FIG. 3, an example method is illustrated by a flowchart 300. At step 302, a first image 10 is acquired via a camera 1a. The first image 10 includes image data of at least a first person in a visible spectrum. At step 304, a second image 20 is acquired via a thermal imaging camera 2a. The second image 20 includes image data of the first person and a temperature reference apparatus 2 in an infrared spectrum. The second image 20, at least in sections has an identical image area as the first image 10.

At step 306, the first image 10 and the second image 20 are superimposed to form a third image 30 based on the identical image area. The third image 30 includes image data of the first image 10 and the second image 20. The superimposing may be performed by matching groups of pixels in the first image 10 and in the second image 20 and/or by a second degree function which assigns pixels of the first image 10 to pixels of the second image 20.

At step 308, a first measurement point is determined within the first image data of the first person of the first image 10. At step 310, a first temperature is determined for the first measurement point based on the third image 30 and the image data of the temperature reference apparatus 2.

At step 312, a fourth image 40 is generated. The fourth image 40 includes image data of the first person and a representation of the first temperature. At step 314, the fourth image 40 is stored when the first temperature is above a threshold temperature. At step 314, when the temperature above a threshold temperature, denying the first person access to a secured area and/or triggering an alarm when the first temperature is above the threshold temperature, at step 316.

At step 318, the first person is identified via a database 4. The database 4 includes identifications of persons and images of the persons. At step 320, a competent authority is identified for the first person via the database 4 and an alarm is triggered at the competent authority, at step 322.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method for detecting a plurality of persons with fever, in view of a plurality of acute infections of the plurality of persons, for access control to a plurality of secure areas, the method comprising the steps:
acquiring a first image by a camera, the first image including a first image data of at least a first person in a visible spectrum;
acquiring a second image by a thermal imaging camera, wherein the second image includes a second image data of the first person and a temperature reference apparatus in an infrared spectrum, and wherein the second image, at least in sections, has an identical image area as the first image;
superimposing the first image and the second image to form a third image based on the identical image area, wherein the third image includes a third image data of the first image and the second image;
determining a first measurement point within the first image data of the at least first person of the first image; and
determining a first temperature for the first measurement point based on the third image and the second image data of the temperature reference apparatus.

2. The method according to claim 1, further comprising the step:
generating a fourth image, the fourth image having a fourth image data of the first person and a representation of the first temperature.

3. The method according to claim 2, further comprising the step:
storing the fourth image when the first temperature is above a threshold temperature.

4. The method according to claim 3, further comprising the steps:
denying the first person access to a secured area in the event that the first temperature is above the threshold temperature, and
triggering an alarm when the first temperature is above the threshold temperature.

5. The method according to claim 4, further comprising the step:
identifying the first person by a database, the database having identifications of persons and images of the persons.

6. The method according to claim 5, further comprising the step:
identifying a competent authority for the first person by the database, and
triggering an alarm at the competent authority.

7. The method according to claim 1, wherein the superimposing is performed by matching groups of pixels in the first image and in the second image or by a second degree function which assigns pixels of the first image to pixels of the second image.

8. The method according to claim 1, wherein the first image includes further persons in addition to the first person, comprising the step performing previous steps for the further persons.

9. An apparatus for detecting a plurality of persons with a fever in view of an acute infection of the plurality of persons for access control for a plurality of secured areas, the apparatus comprising
a camera for capturing a first image, wherein the first image has a first image data of at least a first person in a visible spectrum;
a thermal imaging camera for capturing a second image, wherein the second image includes a second image data of the first person and a temperature reference apparatus in an infrared spectrum, and wherein the second image at least in sections has an identical image area as the first image;
a temperature reference apparatus, wherein the temperature reference apparatus is positioned within an image area of the second image; and
a computing apparatus having a processing device, and a non-transitory, processor-readable storage medium in communication with the processing device, wherein the non-transitory, processor-readable storage medium comprising one or more programming instructions that, when executed, cause the processing device to:
superimpose the first image and the second image to form a third image based on the identical image area, the third image having a third image data of the first image and the second image,
determine a first measuring point within the image data of the first person,
determine a first temperature for the first measuring point on the basis of the third image and the image data of the temperature reference apparatus, and
generate a fourth image, wherein the fourth image includes a fourth image data of the first person and a representation of the first temperature.

10. A computer program product for detecting a plurality of persons with fever, in view of a plurality of acute infections of the plurality of persons, for access control to a plurality of secure areas, the computer program product comprising
a computing apparatus having a processing device and a non-transitory, processor-readable storage medium in communication with the processing device, wherein the non-transitory, processor-readable storage medium comprising one or more programming instructions that, when executed by the computer program product, cause the processing device to:
acquire a first image by a camera, the first image including a first image data of at least a first person in a visible spectrum;
acquire a second image by a thermal imaging camera, wherein the second image includes a second image data of the first person and a temperature reference apparatus in an infrared spectrum, and wherein the second image, at least in sections, has an identical image area as the first image;
superimpose the first image and the second image to form a third image based on the identical image area, wherein the third image includes a third image data of the first image and the second image;
determine a first measurement point within the first image data of the at least first person of the first image; and
determine a first temperature for the first measurement point based on the third image and the second image data of the temperature reference apparatus.
